(19) United States
(12) Patent Application Publication
(10) Pub. No.: US 2022/0265970 A1
(43) Pub. Date: Aug. 25, 2022

(54) MEDICANT DELIVERY APPARATUSES, SYSTEMS, AND METHODS

(71) Applicant: Gyrus ACMI, Inc, d/b/a Olympus Surgical Technologies America, Westborough, MA (US)

(72) Inventor: Alyssa G. Philipps, Seattle, WA (US)

(21) Appl. No.: 17/674,987

(22) Filed: Feb. 18, 2022

Related U.S. Application Data
(60) Provisional application No. 63/151,827, filed on Feb. 22, 2021.

(51) Int. Cl. A61M 25/04; A61M 25/06; A61M 25/01; A61B 1/267; A61B 1/018

(52) U.S. Cl. CPC A61M 25/04; A61M 25/06; A61M 25/0136; A61M 25/0138; A61B 1/2676; A61B 1/018

(57) ABSTRACT

Various disclosed embodiments include illustrative apparatuses, systems, and methods for injecting medicant into target tissue. In an illustrative embodiment, an illustrative apparatus includes a distal end, a proximal end configured to engage a delivery catheter, and a plurality of flexible bands that extend from the distal end to the proximal end. The flexible bands exhibit a delivery configuration and a deployed expanded configuration. When the plurality of flexible bands are in the deployed expanded configuration a cavity is formed and a medicant receivable via the delivery catheter is disbursable into the cavity.

### BACKGROUND

The statements in this section merely provide background information related to the present disclosure and may not constitute prior art.

Currently, when a medicant is injected into target tissue, some of the medicant may leak out of the target tissue. For example, when the medicant is injected into a lung, some of the medicant can backflow along the delivery needle, flow into an airway, or move into other areas of the lung or surrounding organ.

### BRIEF SUMMARY

Various disclosed embodiments include illustrative apparatuses, systems, and methods for injecting medicant into target tissue.

In an illustrative embodiment, an illustrative apparatus includes a distal end, a proximal end configured to engage a delivery catheter, and a plurality of flexible bands that extend from the distal end to the proximal end. The flexible bands exhibit a delivery configuration and a deployed expanded configuration. When the plurality of flexible bands are in the deployed expanded configuration a cavity is formed and a medicant receivable via the delivery catheter is disbursable into the cavity.

In another illustrative embodiment, an illustrative system includes a scope having a working channel, a dual delivery device having a handle and a flexible catheter couplable to the handle, a medicant delivery device couplable to the handle of the dual delivery device, and an expandable cage device. The expandable cage device includes a distal end, a proximal end frictionally engaged with the delivery catheter, and a plurality of flexible bands that extend from the distal end to the proximal end. The flexible bands exhibit a delivery configuration and a deployed expanded configuration. When the plurality of flexible bands are in the deployed expanded configuration a cavity is formed and a medicant receivable via the delivery catheter is disbursable into the cavity.

In another illustrative embodiment, an illustrative method includes inserting a delivery device with an attached cage device in a collapsed mode into a scope, penetrating a distal end of the delivery device with the cage device into target tissue, expanding the cage device within the target tissue, delivering medicant into a cavity created by the expanded cage device, and removing the delivery device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Illustrative embodiments are illustrated in referenced figures of the drawings. It is intended that the embodiments and figures disclosed herein are to be considered illustrative rather than restrictive.

FIG. 1 is a side view of a medical tool.

FIG. 2 is a perspective view of a cage device used with the medical tool of FIG. 1 in a first state.

FIG. 3 is a perspective view of a cage device attached at the distal end of the medical tool of FIG. 1 in a first state.

FIG. 4 is a perspective view of the cage device of FIG. 3 in a second state.

FIG. 5 is a perspective view of a cage device in the second state.

FIG. 6 is a perspective view of a cage device in the second state.

FIG. 7 is a perspective view of a cage device in the second state.

FIG. 8 is a perspective view of a cage device in the second state.

FIG. 9 is a perspective view of a cage device in the first state.

FIG. 10 is a perspective view of a cage device of FIG. 9 in the second state.

FIG. 11 is a flow chart of a method of using the medical tool.

FIG. 12 is a perspective view of a cage device used with the medical tool of FIG. 1 in a first state.

FIG. 13 is a perspective view of the cage device of FIG. 12 in a second state.

Like reference symbols in the various drawings generally indicate like elements.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various disclosed embodiments include illustrative apparatuses, medical systems, and methods for delivering medicant into target tissue.

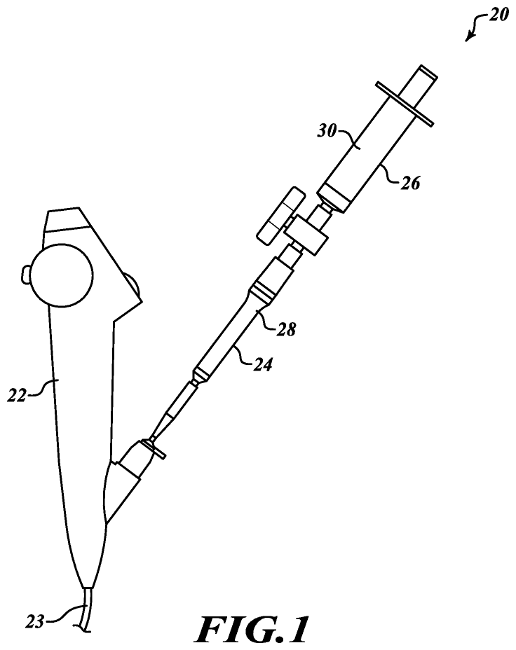

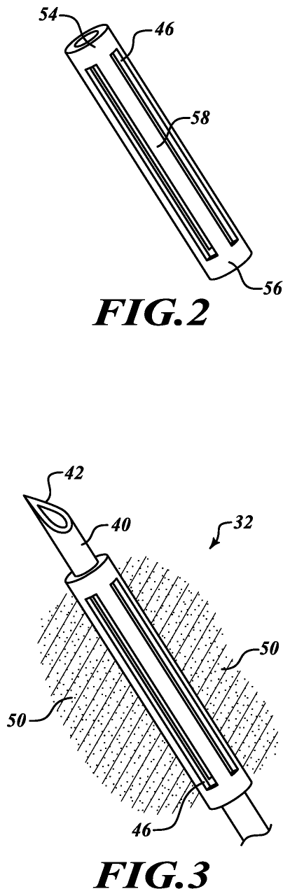

Referring now to FIG. 1 and given by way of overview, in various embodiments, an illustrative medicant delivery system 20 includes a scope 22, a dual delivery device 24, and a medicant delivery device 26. The scope 22 may include multi-use or single-use endoscopes, such as bronchoscopes, laparoscopes, laryngoscopes, or the like. The dual delivery device 24 is received within an access port of the scope 22. The medicant delivery device 26 is attachable to an end of a handle 28 of the dual delivery device 24. The dual delivery device 24 includes a flexible delivery catheter (not shown in FIG. 1) connected to the handle 28, both of which include a lumen (working channel) for delivery of a medicant inserted by the medicant delivery device 26. The dual delivery device 24 is capable of delivering an expandable cage (not shown) into target tissue, as will be described in more detail below with reference to FIGS. 2-8.

The medicant delivery device 26, such as a syringe or the like, includes a housing 30 having a reservoir for containing a medicant fluid or gel and a movable piston (not shown) used to force the medicant fluid or gel into the lumens of the dual delivery device 24.

Referring additionally to FIG. 2, in various embodiments, an expandable cage device 46 is created from a medical grade hypotube that is machined to include multiple expandable bars 58 that extend between a proximal ring 56 and a distal ring 54. Referring additionally to FIG. 3, a flexible delivery device 40 of the dual delivery device 24 includes a distal end 32 that receives the expandable cage device 46 in a collapsed configuration in a delivery mode of operation. The very distal end of the flexible delivery device 40 may include a sharpened tip 42, such as a Huber tip needle. The rings 54 and 56 include an inner diameter 59 sized to slidably receive the flexible delivery device 40. It will be appreciated that the expandable cage device 46 may be created by applying laser cuts to a medical grade metal, such as nitinol. The expandable cage device 46 is deployable within target tissue 50, as further described below. The flexible delivery device 40 with the expandable cage device 46 may be received within a catheter or sheath (not shown), thus protecting the working channel of the bronchoscope during delivery.

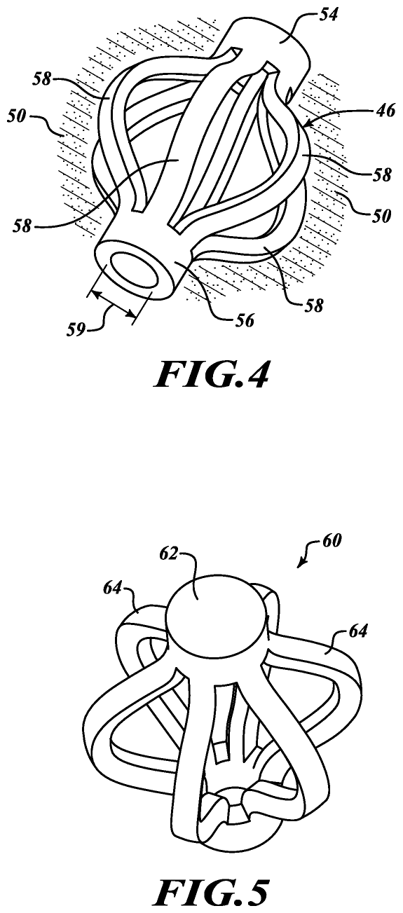

Referring additionally to FIG. 4, in various embodiments the expandable cage device 46 is shown in an expanded configuration within the target tissue 50. In various embodiments, in the expanded configuration, the expandable bars 58 may assume a curved configuration as the expandable bars 58 expand away from a longitudinal axis 59 of the expandable cage device 46, thus creating a cavity within the expandable bars 58. When in the expanded configuration, rings 54 and 56 are moved closer longitudinally to each other than when the expandable cage device 46 is in the collapsed configuration. Once the flexible delivery device 40 with the attached expandable cage device 46 is inserted into the target tissue 50, the expandable cage device 46 assumes the expanded configuration. The expandable cage device 46 may be shape-set to achieve the expanded configuration when exposed to temperatures expected at the target tissue 50. Thus, the expandable cage device 46 automatically expands when in the target tissue 50. Specifically, in response to exposure to the expected temperatures at the target tissue 50, the expandable bars 58 expand outwardly from the rings 54 and 56 and draw the rings 54 and 56 closer together. It will be appreciated that the expandable cage device 46 may also be placed in the expanded configuration by use of a balloon (not shown) included with the flexible delivery device 40 and situated within the expandable cage device 58. As a result, inflation of the balloon would cause the expandable bars 58 to expand outwardly and draw the rings 54 and 56 closer together.

During a procedure to deliver a medicant into target tissue, such as a tumor, lesion, lymph node, or the like, the expandable cage device 46 is attached to the distal end 32 of the flexible delivery device 40 in the collapsed configuration. The flexible delivery device 40 and the expandable cage device 46 are passed through the handle 28 and a flexible insertion tube 23 of the scope 22, then out an exit port of the flexible insertion tube 23 and into the target tissue 50.

Referring to FIG. 5, in various embodiments, an expandable cage device 60 includes a solid distal cap 62 attached to distal ends of expandable bars 64. A delivery catheter (not shown) of a dual delivery device may include a blunt end for engaging with a proximal surface of the distal cap 62.

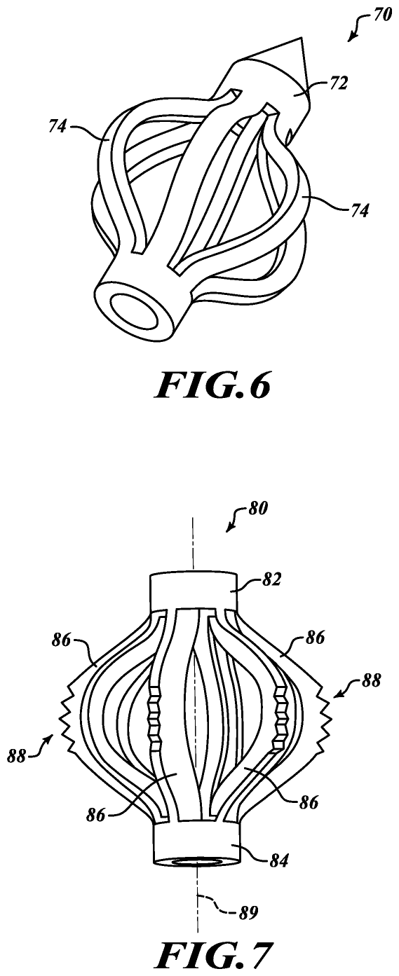

Referring to FIG. 6, in various embodiments, an expandable cage device 70 includes a sharpened pointed distal tip 72 attached to distal ends of expandable bars 74. A delivery catheter (not shown) of the dual delivery device 24 may include a blunt end for engaging with a proximal surface of the distal tip 72.

Referring to FIG. 7, in various embodiments, an expandable cage device 80 includes a distal end 82, a proximal end 84, and multiple expandable bars 86 extending between the ends 82 and 84. An outer surface of one or more of the expandable bars 86 includes one or more sharpened ridges 88. The sharpened ridges 88 may be formed by molding or cutting grooves into the surface of the expandable bars 86. The grooves are perpendicular to a longitudinal axis 89 of the expandable cage device 80 to form the sharpened ridges. The sharpened ridges 88 cut into tissue during expansion of the cage device 80.

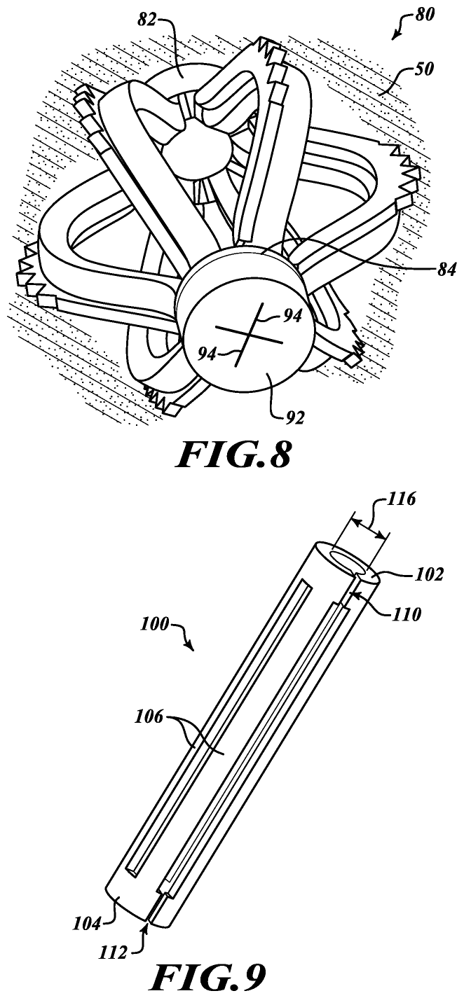

In various embodiments, as shown in FIG. 8, the expandable cage device 80 includes a self-sealing valve 92 located at the proximal end 84. The self-sealing valve 92 is formed of a flexible medical grade material, such as silicone or the like. The self-sealing valve 92 may be friction fitted over the proximal end 84 and/or attached to the proximal end 84 with an adhesive and/or overmolded to the proximal end 84. The self-sealing valve 92 includes slots 94 that allow the material of the self-sealing valve 92 to deflect and receive the flexible delivery device 40 (FIG. 3) of the delivery device 24 (FIG. 1) during delivery of the expandable cage device 80. Once the expandable cage device 80 has been inserted into the target tissue 50, the expandable cage device 80 is expanded as shown, then the flexible delivery catheter is retracted during or after medicant is delivered inside the expanded cage device 80. After a desired amount of medicant has been delivered via the flexible delivery device 40, the flexible delivery device 40 is retracted proximal from the expandable cage device 80, whereby the self-sealing valve 92 closes, thus blocking medicant from escaping. The self-sealing valve 92 may be used with any of the other illustrative cage devices. The cage device 80 remains in the target tissue for allowing possible access at a later time.

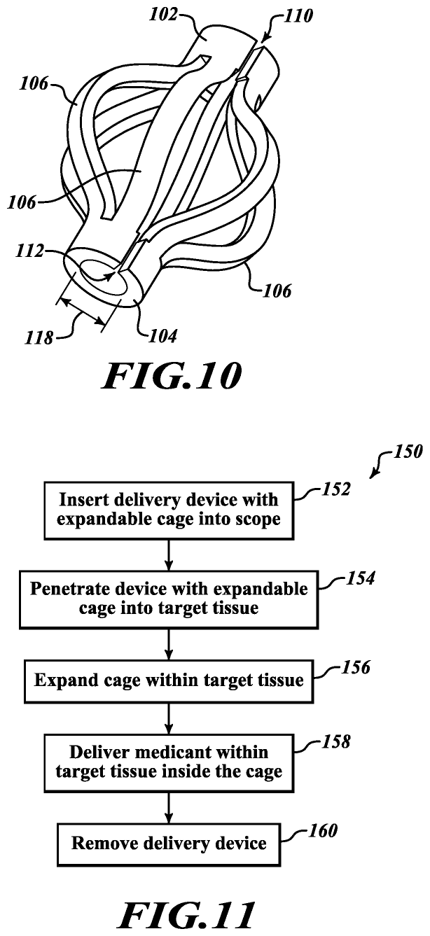

Referring additionally to FIGS. 9 and 10, an expandable cage device 100 includes a distal end 102, a proximal end 104, and multiple expandable bars 106 extending between the ends 102 and 104. The ends 102 and 104 are generally ring-shaped. The ends 102 and 104 include longitudinal slots 110 and 112, respectively. The slots 110 and 112 allow the ends 102 and 104 to expand radially. The ends 102 and 104 include longitudinal holes having a first inner diameter 116 when the expandable cage device 100 is in the collapsed configuration (FIG. 9) and a second inner diameter 118 when the expandable cage device 100 is in the expanded configuration (FIG. 10). The second inner diameter 118 is greater than the first inner diameter 116. When the expandable cage device 100 is in the collapsed configuration, the ends 102 and 104 with the first inner diameter 116 provides enough friction with the flexible delivery device 40 (FIG. 3) for maintaining attachment of the expandable cage device 100 to the flexible delivery device 40 during delivery through the scope. Once the expandable cage device 100 is located within the target tissue, the multiple expandable bars 106 and the ends 102 and 104 of the expandable cage device 100 expand. The holes of expanded ends 102 and 104 now have the second inner diameter 118. With second inner diameter 118 the ends 102 and 104 apply little or no frictional force on the flexible delivery device 40, thus allowing the flexible delivery device 40 to be slidably removed while leaving the expandable cage device 100 in place. The diameters 116 and 118 may be set during a shape set procedure.

Referring additionally to FIG. 11, in various embodiments an illustrative method 150 is provided for delivering medicant within target tissue of a patient. At a block 152, the delivery device 40 with the expandable cage device 46 is inserted into the scope 22. At a block 154, the distal end of the delivery device 40 and the expandable cage device 46 are inserted into the target tissue 50. At a block 156, the expandable cage device 46 is expanded within the target tissue 50. At a block 158, medicant is delivered through the delivery device 40 within the target tissue 50 and the expandable cage device 46. At a block 160, the delivery device 40 without the expandable cage device 46 and the scope 22 are then fully removed from the patient.

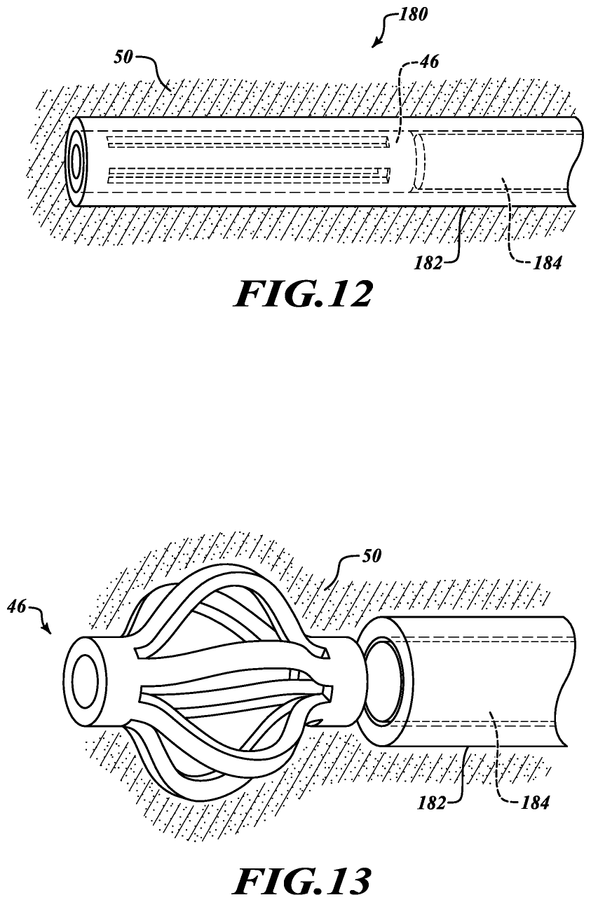

Referring additionally to FIGS. 12 and 13, in various embodiments the expandable cage device 46 is delivered within a catheter 182 into the target tissue 50. Once the catheter 182 with the expandable cage device 46 is properly located within the target tissue 50, the catheter 182 is retracted while a stylet 184 located proximally from the expandable cage device 46 maintains position relative to the target tissue 50. The retracting catheter 182 expels the expandable cage device 46 into the target tissue 50. Once the expandable cage device 46 is expelled, the expandable cage device 46 transforms to the expanded configuration (FIG. 3).

From the foregoing discussion and associated drawing figures, it will be appreciated that various embodiments have been disclosed and illustrated. To that end and without any implication of any limitation (which is not to be inferred), the following paragraphs set forth non-limiting summaries of various embodiments disclosed herein by way of example only and not of limitation:

A. An apparatus comprising: a distal end; a proximal end configured to engage the delivery catheter; and a plurality of flexible bands configured to extend from the distal end to the proximal end, the flexible bands configured to exhibit a delivery configuration and a deployed expanded configuration, wherein when the plurality of flexible bands are in the deployed expanded configuration a cavity is formed and a medicant receivable via the delivery catheter is disbursable through the cavity.

B. The apparatus of A, wherein the flexible bands are shape set to exhibit the deployed expanded configuration at a predefined temperature, the flexible bands have a curved configuration away from a longitudinal axis of the apparatus in the deployed expanded configuration.

C. The apparatus of A, wherein the distal end includes a distal portion having a cross-sectional dimension that reduces in value in a distal direction.

D. The apparatus of C, wherein the distal portion of the distal end includes a tissue piercing tip.

E. The apparatus of A, further comprising a self-sealing membrane disposed at the proximal end.

F. The apparatus of A, wherein at least one of the flexible bands includes a tissue cutting ridge disposed on an outer side of the flexible band.

G. The apparatus of F, wherein the tissue cutting ridge is approximately perpendicular to a longitudinal axis of the apparatus.

H. The apparatus of A, wherein at least one of the distal end or the proximal end is configured to expand radially when the plurality of flexible bands are in the deployed expanded configuration.

I. The apparatus of H, wherein at least one of the distal end or the proximal end includes a longitudinal slot.

J. A system comprising: a scope having a working channel; a dual delivery device including a handle and a flexible catheter couplable to the handle; a medicant delivery device couplable to the handle of the dual delivery device; and an expandable cage device including: a distal end; a proximal end configured to frictionally engage the delivery catheter; and a plurality of flexible bands configured to extend from the distal end to the proximal end, the flexible bands configured to exhibit a delivery configuration and a deployed expanded configuration, wherein when the plurality of flexible bands are in the deployed expanded configuration a cavity is formed and a medicant receivable via the delivery catheter is disbursable through the cavity.

K. The system of J, wherein the flexible bands are shape set to exhibit the deployed expanded configuration at a predefined temperature, the flexible bands have a curved configuration away from a longitudinal axis of the apparatus in the deployed expanded configuration.

L. The system of J, wherein the distal end includes a distal portion having a cross-sectional dimension that reduces in value in a distal direction.

M. The system of L, wherein the distal portion of the distal end includes a tissue piercing tip.

N. The system of J, wherein the expandable cage device further includes a self-sealing membrane disposed at the proximal end of the expandable cage device.

O. The system of J, wherein at least one of the flexible bands includes a tissue cutting ridge disposed on an outer side of the flexible band.

P. The system of O, wherein the tissue cutting ridge is approximately perpendicular to a longitudinal axis of the expandable cage device.

Q. The system of J, wherein at least one of the distal end or the proximal end includes a longitudinal slot configured to allow the distal end or the proximal end to expand radially when the plurality of flexible bands are in the deployed expanded configuration.

R. A method comprising: inserting a delivery device into a scope, the delivery device including a cage device in a collapsed mode; penetrating a distal end of the delivery device with the cage device into target tissue; expanding the cage device within the target tissue; delivering medicant into a cavity created by the expanded cage device; and removing the delivery device.

S. The method of R, wherein the expanding includes expanding the cage device to a shape set configuration in response to a temperature of the target tissue.

T. The method of S, wherein removing the delivery device includes sealing a proximal end of the expandable cage device in response to the distal end of the delivery device being withdrawn from the expandable cage.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (for example “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

What is claimed is:

1. An apparatus comprising:
a distal end;
a proximal end configured to engage a delivery catheter; and
a plurality of flexible bands configured to extend from the distal end to the proximal end, the flexible bands configured to exhibit a delivery configuration and a deployed expanded configuration, wherein when the plurality of flexible bands are in the deployed expanded configuration a cavity is formed and a medicant receivable via the delivery catheter is disbursable into the cavity.

2. The apparatus of claim 1, wherein the flexible bands are shape set to exhibit the deployed expanded configuration at a predefined temperature, the flexible bands have a curved configuration away from a longitudinal axis of the apparatus in the deployed expanded configuration.

3. The apparatus of claim 1, wherein the distal end includes a distal portion having a cross-sectional dimension that reduces in value in a distal direction.

4. The apparatus of claim 3, wherein the distal portion of the distal end includes a tissue piercing tip.

5. The apparatus of claim 1, further comprising a self-sealing membrane disposed at the proximal end.

6. The apparatus of claim 1, wherein at least one of the flexible bands includes a tissue cutting ridge disposed on an outer side of the flexible band.

7. The apparatus of claim 6, wherein the tissue cutting ridge is approximately perpendicular to a longitudinal axis of the apparatus.

8. The apparatus of claim 1, wherein at least one of the distal end or the proximal end is configured to expand radially when the plurality of flexible bands are in the deployed expanded configuration.

9. The apparatus of claim 8, wherein at least one of the distal end or the proximal end includes a longitudinal slot.

10. A system comprising:
a scope having a working channel;
a dual delivery device including a handle and a flexible catheter couplable to the handle;
a medicant delivery device couplable to the handle of the dual delivery device; and
an expandable cage device including:
a distal end;
a proximal end configured to frictionally engage the delivery catheter; and
a plurality of flexible bands configured to extend from the distal end to the proximal end, the flexible bands configured to exhibit a delivery configuration and a deployed expanded configuration, wherein when the plurality of flexible bands are in the deployed expanded configuration a cavity is formed and a medicant receivable via the delivery catheter is disbursable into the cavity.

11. The system of claim 10, wherein the flexible bands are shape set to exhibit the deployed expanded configuration at a predefined temperature, the flexible bands have a curved configuration away from a longitudinal axis of the apparatus in the deployed expanded configuration.

12. The system of claim 10, wherein the distal end includes a distal portion having a cross-sectional dimension that reduces in value in a distal direction.

13. The system of claim 12, wherein the distal portion of the distal end includes a tissue piercing tip.

14. The system of claim 10, wherein the expandable cage device further includes a self-sealing membrane disposed at the proximal end of the expandable cage device.

15. The system of claim 10, wherein at least one of the flexible bands includes a tissue cutting ridge disposed on an outer side of the flexible band.

16. The system of claim 15, wherein the tissue cutting ridge is approximately perpendicular to a longitudinal axis of the expandable cage device.

17. The system of claim 10, wherein at least one of the distal end or the proximal end includes a longitudinal slot configured to allow the distal end or the proximal end to expand radially when the plurality of flexible bands are in the deployed expanded configuration.

18. A method comprising:
inserting a delivery device into a scope, the delivery device including a cage device in a collapsed mode;
penetrating a distal end of the delivery device with the cage device into target tissue;
expanding the cage device within the target tissue;
delivering medicant into a cavity created by the expanded cage device; and
removing the delivery device.

19. The method of claim 18, wherein the expanding includes expanding the cage device to a shape set configuration in response to a temperature of the target tissue.

20. The method of claim 19, wherein removing the delivery device includes sealing a proximal end of the expandable cage device in response to the distal end of the delivery device being withdrawn from the expandable cage.
